(19) United States
(12) Patent Application Publication
(10) Pub. No.: US 2015/0051673 A1
(43) Pub. Date: Feb. 19, 2015

(54) DEVICE AND METHOD FOR CONTROLLING, EITHER INCREASING OR DECREASING, THE BODY TEMPERATURE OF A PATIENT

(71) Applicant: Rodrigo Rivas Tapia, Santiago (CL)

(72) Inventor: Rodrigo Rivas Tapia, Santiago (CL)

(21) Appl. No.: 14/364,021

(22) PCT Filed: Dec. 6, 2011
(86) PCT No.: PCT/CL2011/000073

§ 371 Date: Nov. 5, 2014

(30) Foreign Application Priority Data
Aug. 18, 2011 (CL) 2030-2011

(51) Int. Cl. A61F 7/00

(52) U.S. Cl. CPC A61F 7/0097; A61F 2007/0011; A61F 2007/0055; A61F 2007/0054; A61F 2007/0091; A61F 2007/0094
USPC 607/107; 607/104

(57) ABSTRACT

Noninvasive system for rising or lowering a patient's body temperature in a controlled manner comprising: a) a cervical blanket (1) with ducts (2) through which a fluid circulates (3); b) a fluid (3) capable of transferring heat from and to external bodies; c) a heat exchanger (4) which rises or lowers the fluid temperature that circulates through cervical blanket ducts; d) means of connection (5) between ducts (2) of the cervical blanket (1) an the heat exchanger (4); e) a body temperature sensor (6); f) a controller (7) which rises or lowers fluid temperature according to a predetermined temperature and according to the temperature measured by the body temperature sensor (6).

The present invention relates to a device and method for controlling, either by rising or lowering, the body temperature of a medical patient. Particularly, the invention is applied in the treatment of patients with hypothermia requiring rising body temperature in a controlled fashion, as well as lowering the patient's temperature in case of therapeutic hypothermia.

### BACKGROUND OF THE INVENTION

Human beings are homothermous animals, i.e. they are able to regulate their own temperature, which means that under normal physiological conditions they maintain a constant body temperature and within a very narrow range, between 36.6+/−0.38° C., despite large oscillations at room temperature. This temperature is maintained by a balance between heat production and loss.

Main heat production sources are: i) heat generation by biochemical processes that occur in cells of our body and, ii) heat generation by chemical reactions produced inside the muscles upon contraction. In turn, heat loss sources are: i) radiation, which is the way objects with a temperature above absolute zero lose heat, ii) conduction, which is when a body with a given temperature is in direct contact with a cooler body so that part of the heat thereof is transferred; iii) convection, is the transfer of heat from the body to the air or water particles which are in contact therewith, wherein these particles are heated when are in contact with the body surface and, subsequently, when they leave this surface, it is occupied by other cooler particles which in turn are heated, and so on, and iv) evaporation, corresponds to heat loss through sweating which evaporates from the skin causing heat loss.

There exists a need for body temperature control within the clinical setting.

In the case of patients in intensive Care Units (ICU), they undergo long surgical procedures. Likewise, patients rescued from the accident zone, suffer from heat loss, altering the balance between heat production and loss, either because at ICU patient clothes are removed so that the skin is exposed to punctures, inspections or surgeries, and where the skin is washed with cold liquids, or because cold liquid injections are administered to patients. In the case of accident injured patients exposed to the environmental conditions and lying on cold places (pavement or floor) with their clothes wet with water or blood who are subsequently rescued, their clothes are removed in order to inspect them and to identify and assess the bleeding sites. Many are already cold when the rescue unit arrives. Both groups of patients lose heat by radiation, conduction and convection and together with the inability to move due to unconsciousness (caused by serious head traumas or by general anesthesia) they neither can generate heat by muscle contraction nor can keep warm in order to lose less heat by radiation. The natural consequence is hypothermia.

Accidental hypothermia is defined as body temperature below 35° C. However, regardless of achieving those levels of temperature, any degree of cooling may be detrimental for patients.

This body cooling involves a number of consequences. Coagulation alterations leading to an increased bleeding in patients; the central nervous system is affected resulting in somnolence and the immune system is also affected making patients susceptible to infections. Within the cardiovascular system, due to the intense reaction of the organism to keep and recover heat, arterial hypertension and tachycardia occur in patients with risk factors (elderly patients with a history of heart diseases), may result in a cardiac arrest.

On the other hand, under other conditions, such as the case of those patients suffering a cardiac arrest (with absence of brain blood flow) or asphyxia, the only efficacious measure to be taken in order to stop or limit the area of brain cell death caused by the lack of oxygen is that patients undergo a cooling process as soon as possible (during a period of less than 6 hours), specifically, to achieve a hypothermia state at a temperature of 32° C. This is known as therapeutic hypothermia.

By achieving this temperature in patients, and specifically in their brains, the subsequent consequences associated with this kind of accidents are milder than would otherwise have been the case. The mortality of patients suffering a cardiac arrest that undergo hypothermia is lower than that of those treated at normal temperature.

Hypothermia is a global standard treatment tool, for treating newborns suffering from neonatal asphyxia. It is easy to implement in them because they are small, have larger body surface areas exposed relative their weight from where the heat is lost (compared to adults) and they tend to cool rapidly in a spontaneous way. In turn, although the benefits of this measure are clear, the implementation thereof, (i.e., the methods for lowering temperature), are not clearly established in adults.

With regard to the ways used to modify a patient's body temperature, there exist devices designed to exchange heat with a patient by making use of the principle of convection.

A particular type of convection is air convection, which allows cold and heat transfer through generation of air currents at a set temperature in a closed system that surrounds a patient. Through this technique, good results with regard to heat preservation and temperature rising under hypothermia conditions are obtained, but since large air-inflatable sheets made of light material located over the patient's skin exposed area are required, the body inspection which is vital in serious injured patients is obstructed.

On the other hand, in patients that undergo major surgeries, wherein the abdomen, thoracic cavity, or plastic or aesthetic surgery reconstruction are involved and wherein hypothermia occurs as a consequence of the exposure of large areas of the body at room temperature, the use of this system for preventing or treating hypothermia is confined to the lower “available”, extremities, however, they are not that effective for capturing and transferring heat towards the whole body. As a reflex form, the extremities exposed to room air experience a reduced blood flow towards the surface, i.e., skin, tips of the toes, etc., due to vasoconstriction phenomena to avoid heat loss. Therefore, by applying an air convection heater to the extremities; the blood being the vehicle that transports this change of temperature to the rest of the body; and, as a result of the cold, the amount of blood that circulates superficially in the extremity decreases, only the skin can be warmed, and the rise, in temperature can not be transferred to the rest of the body, or if this is achieved, it occurs at a very low speed.

The devices that use air convection together with these methods, i.e., cool air ventilated mattresses located over the body are not effective to induce hypothermia.

There also exist systems that use water convections. For example, in heart surgery with extracorporeal circulation, a basic premise is to stop the heart and withdraw the blood for the period that the surgery thereon lasts, this surgery either being coronary artery surgery wherein arteries become blocked resulting in cardiac arrests or cardiac valve surgery wherein in certain diseases arteries become so damaged that they have to be changed. Heart is stopped, and blood is withdrawn from vena cava to the ECC machine wherein CO₂ is removed and replaced with oxygen to be returned to the aorta artery. As a consequence of this process, heat from blood is lost resulting in hypothermia.

In the case of a surgery with ECC, since the heart is stopped and does not receive any blood, the patient may suffer damages or may die if they do not receive enough oxygen. To overcome these problems, surgeons, along with heart stoppage, try to make sure that heart is cooled at temperatures ranging from 28 to 32° C. so that the heart can survive around 2 hours without receiving oxygen flow. A water convection temperature exchanger is used to keep the patient at this temperature. Blood is transported from the patient through tubes made of synthetic plastic material to the ECC machine, which in turn comprises a closed container containing circulating water through which piping containing blood passes through without being in direct contact with water but experiencing temperature changes according to the circulating water temperature. Inside this closed container water is circulated at varying temperatures according to what is determined by the heat exchange controllers, it is the most efficient water convection temperature transfer system currently available, capable of cooling patients to 10° C. if required. Once the procedure is completed, and in order to remove patient from ECC, the patient's temperature must be rised, from the temperature during surgery to the normal temperature of approximately 37° C.

This is the most effective method for warming or cooling patient but the usefulness thereof is limited to the field of cardiac surgery due to the degree of invasion for the patient which is not devoid of complication, including death.

There exist noninvasive devices (i.e., devices that do not penetrate patient's anatomical barriers) based on the same principle, which consist of a hollow mattress where the patient lies down; the mattress being made of plastic material within which cold or hot water is circulated but it is not effective for heat transfer in adults or children and it is only used to cool down newborns as a neonatal asphyxia recovery therapy.

### PREVIOUS ART

US20050574723 describes a device for rising temperature at a specific site by radiation. This device comprises layers of flexible material and insulating material. According to this document, the device can he in the form of a blanket and the use thereof in therapeutic settings is disclosed. However, the effect achieved corresponds only to a local temperature rise and the goal thereof is to reduce pain associated with a specific area. The present invention does not relate to patient's local temperature modification, it is used to rise or lower body temperature of a patient in a controlled manner for therapeutic purposes.

US20050248939 describes a system and method for producing therapeutic hypothermia. The system comprises a controller and several garments, wherein garments are connected to a controller (battery operated console). The controller supplies cold fluids to garments so that the body surface is cooled down by such garments. Specifically, the garments described in this document correspond to a kind of helmet capable of lowering temperature, preferably at the patient's head. Unlike this document, the present invention is capable of rising or lowering the patient's body temperature in a controlled manner and it is not limited to the patient's head temperature lowering.

WO2001US22036 describes a medial device capable of removing a large amount of heat from a zone close to the patient's carotid arteries. An endotracheal device that can be used together or separately is also mentioned in this document. In particular, it allows the patient's head temperature lowering while the present invention is capable of controlling the patient's body temperature without limiting to an increment or reduction, and without limiting to a specific body area.

GB20070004465 describes a flexible sheet comprising ducts through which fluids circulate. Such ducts are connected to a temperature controller. Among different uses, the invention is mentioned as a blanket for warming up or cooling down a patient requiring body temperature local or general control. In contrast, a specific form of the blanket and the specific location thereof together with a temperature sensor capable of properly controlling the patient's temperature arc considered in the present invention.

US20060419186 describes a patient warming system. It comprises a covering that may cover the whole torso and neck as well as areas from other parts of the body. It is also mentioned that the system may be selectively activated per zones depending on the capacity of the body zone to capture heat. In contrast, the present invention corresponds to a cervical blanket wherein body temperature can be controlled either by increasing or reducing temperature based on the therapeutic requirements.

WO2010US00400 describes a whole body blanket that can be inflated with warmed air for patient warming. The structure of the blanket allows a selective activation so that specific parts of the body are exposed to heat. The present invention relates to a small blanket located at the patient's cervical zone and allows controlling of the patient's body temperature either by rising or lowering temperature, for example, in the case of therapeutic hypothermia.

To the best of the applicant knowledge, the aforementioned apparatuses, devices and methods of the previous art relate to the temperature rising, either locally or corporal, or to the temperature lowering, wherein documents focusing on lowering the patient's head temperature stand out. On the contrary, the present invention corresponds to a body temperature control system that can be used, for example, in cases of therapeutic hypothermia, wherein patient's temperature is lowered in the first place, and then when the clinical goal is achieved; the patient is helped to recover the appropriate body temperature. The control of body temperature and not only local application of cold and heat, sets us apart from the other prior art alternatives.

### BRIEF DESCRIPTION OF THE INVENTION

The present invention corresponds to a noninvasive system for rising or lowering body temperature of a subject in a controlled manner, wherein temperature rising or lowering are carried out according to a predetermined temperature and according to the temperature measured by a body temperature sensor.

The present invention also considers a method for controlling the body temperature of a patient, either by rising or lowering temperature.

### BRIEF DESCRIPTION OF THE FIGURES

FIG. 1 shows an schematic view of the system of the present invention comprising a cervical blanket (1) together with ducts (2) through which a fluid circulates (3); a fluid (3) capable of transferring heat from and to external bodies; a heat exchanger (4) which rises or lowers the temperature of the fluid circulating through the ducts (2) of the cervical blanket (1); means of connection (5) between the cervical blanket ducts and heat exchanger (4); a body temperature sensor (6); and a controller (7).

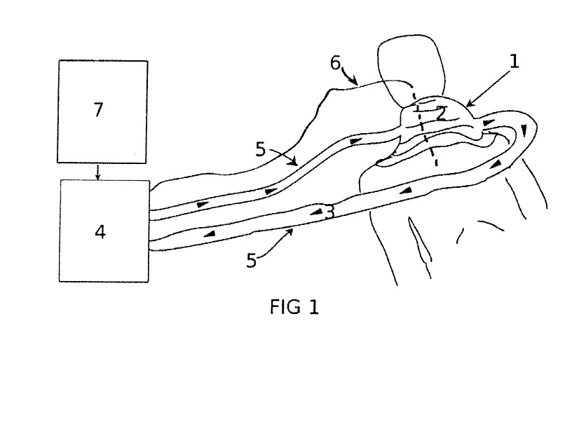

FIG. 2 shows a particular embodiment of the cervical blanket, when the fluid (3) is air. A) Between the internal and external layer there exist septa (8) (made of the same material as the internal layer membrane) with septal fenestrations (9) travelling in a cross-sectional manner across the cervical blanket in order to achieve a homogeneous distribution of the propelled fluid. B) the internal sheet, at the external surface thereof which will be in contact with the patient's skin, comprises two self-adhesive bands (11) arranged lengthwise and that adhere to the patient's skin and further comprising fenestrations (10) or vents that enable air to flow to the patient's skin to improve convection.

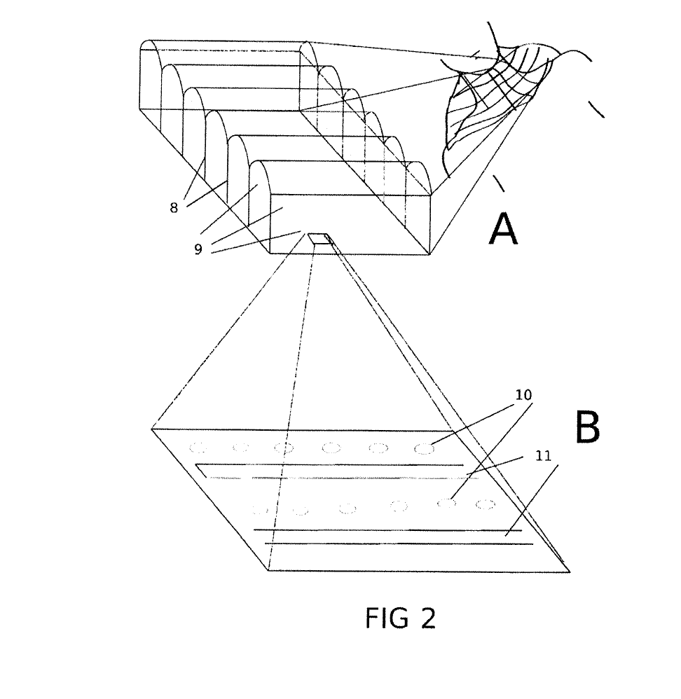

FIG. 3 shows an optional embodiment of the system of the present invention, when the fluid is air, wherein from the edge of the cervical blanket (1) that is in contact with the patient's jaw, a veil (12) made of transparent impermeable material covering the entire head upwards is projected.

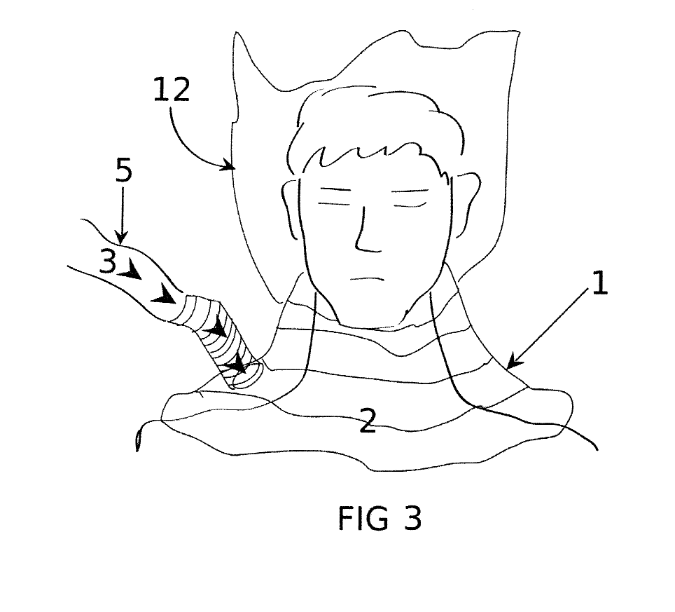

FIG. 4 shows in detail a second embodiment of the cervical blanket, when the fluid used is liquid. A) a hollow, plastic, tubular structure is observed; the structure comprising ducts (2) through which the liquid fluid (3) circulates and comprises a fluid inlet (14) that receives the fluid (3) from the heat exchanger (4), and an outlet (15) which transports the fluid (3) to the heat exchanger, wherein the inlet connection means is subdivided into a series of ramifications corresponding to the ducts (2) through which the fluid (3) circulates until reaching the liquid fluid outlet zone where they converge into a single duct which returns the fluid to the heat exchanger. In a more particular embodiment, there exists a vacuum line (13) which enables the cavity between the cervical blanket and patient's skin to be subjected to constant aspiration to cause vacuum. This causes that both walls of this cavity to be in close proximity until no space is left between each other and they become fixed adopting the shape of the contacting surface. B) A cross-sectional view of the cervical blanket is observed (1) over a patient and a zone amplification. Within this view the ducts are shown (2) through which the fluid circulates, a virtual space (16), the zone where vacuum (17) is applied between the internal sheet (18) of the cervical blanket (1) and the patient's skin. The internal structure (tubular) which is deflated, i.e. empty, is rather a septal structure like a bee hive, i.e., each tube continues at the neighbor wall. Once expanded through the fluid, they give the blanket the shape of a crust covering the neck with these structures (2).

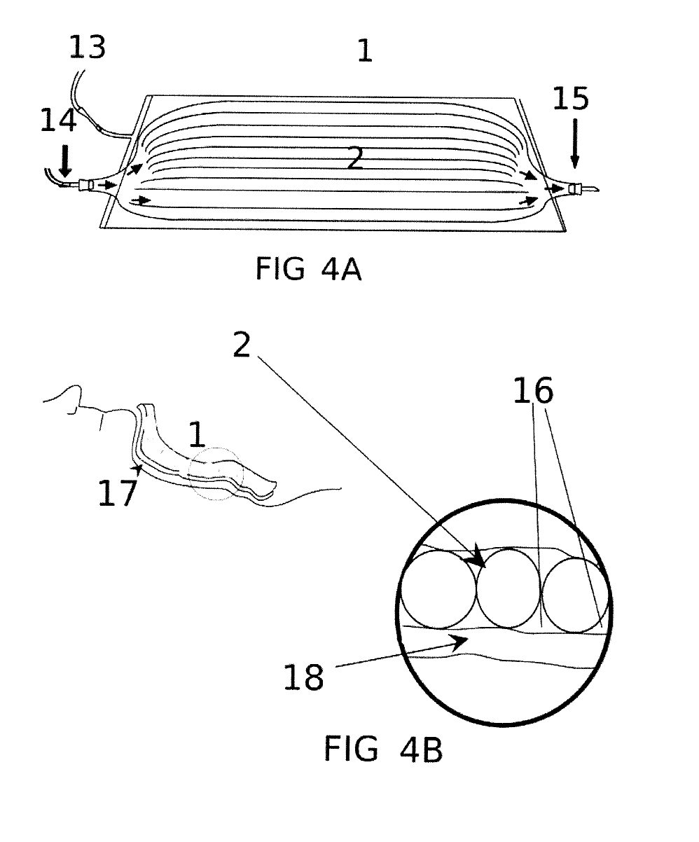

Within the context of the present invention, body temperature should be understood as a person's core temperature. This is because the present invention relates to whole body temperature modification and not to localized and restricted temperature limited to a part of the person's body.

The present invention is based on the application of convection principle, i.e., heat transfer through a fluid in motion which is either air or water, but applied to the neck of the patient.

From the anatomical point of view, neck is a favorable site where convection mechanisms for temperature transfer can be applied, either through water or air, because: i) the blood flow to the head is constant and does not comprise the variations of blood flow to the extremities caused by vasoconstriction phenomena; ii) blood vessels that transport blood to the brain, travel superficially through the whole neck, iii) carotid arteries run 1 cm below the skin surface and jugular veins are exposed in the neck surface; iv) the amount of blood pumped per minute from the heart of an adult at rest is 5 liters, 1 liter of which travels to the head; v) the neck skin (dermis and epidermis) is generally thinner than in other anatomic areas such as, for example, abdomen, thorax. If heat or cold is applied through air or water convection to the neck, the temperature of air or water is quickly transferred, first to the skin and then to the neck tissues comprising vessels through which blood travels to the brain and travels back from the brain. If we know that within a minute more than 20% of the blood pumped from the heart passes through the neck to the brain through carotid veins and travels back through jugular veins form brain to heart, within 5 minutes a blood flow of 5 liters will have passed both ways through the zone where the temperature is applied, travelling upstream to the brain through the artery and travelling back through the vein.

Consequently, the design of a system of heat application through convection, either through the air or through a liquid fluid confined to the cervical or brain area is effective to transfer temperatures to the patient.

In one embodiment, the present invention corresponds to a noninvasive system for rising or lowering body temperature of a person in a controlled manner, comprising a cervical blanket (1) with ducts (2) through which a fluid circulates (3); a fluid (3) capable of transferring heat from and to external bodies; a heat exchanger (4) which rises or lowers the temperature of the fluid circulating inside the ducts (2) of the cervical blanket (1); connection means (5) between the cervical blanket ducts and the heat exchanger (4); a body temperature sensor (6); and a controller (7) which rises or lowers the fluid temperature according to a predetermined temperature and according to the temperature measured by the body temperature sensor.

The present invention also considers a method for controlling a patient's body temperature either by rising or lowering temperature.

In a particular embodiment, the cervical blanket (1) is a disposable inflatable blanket. It is disposable by being a device made of lightweight material (very light), which will be in contact with the patient's skin, thereby being a source of microorganism and infection transmission among patients. The cervical blanket (1) may be manufactured with two thin, synthetic sheets made of latex-free hypoallergenic material, fixed together along the free edge thereof so that between them there exists a virtual space, wherein both thin sheets are in a rectangular shape and once inflated through air conditioning, they adapt to the patient's neck as follows. Particularly, one of the sheets corresponds to the “internal” sheet which is the sheet that is in contact with patient's skin and it is made of a mixture of cellulose and a plastic polymer which renders a more resistant material. The internal sheet whose external surface will be in contact with patient's skin comprises self-adhesive band arranged lengthwise and that adhere to the patient's skin. This layer also comprises fenestrations (10) or vents that enable air to flow to the patient's skin to improve convection.

In particular, the cervical blanket is adhered to the patient's skin at the zone delimited downstream by both clavicles, laterally, by a straight line running across the skin from the acromioclavicular joint to the mastoid apophysis of the temporal bone on both sides, and upperly, by the lower edge of the mandible bone.

The “external” thin layer which is in the same shape as the internal sheet, is a thin layer made of polyethylene with an appendix which emerges from the device lateral surface relative to neck with a conical adaptor where a corrugated tube coming from the heat exchanger (4) is connected. Both sheets, internal and external, are attached not only along the entire free edge thereof but also in a cross-sectional manner via perpendicular sheets attached to both sheet so that incomplete septa (8) (fenestrated (9)) are produced so the air travels freely across the whole length of both sheets serving as an anchor to prevent the device, once inflated, from having a globular shape.

The cervical blanket (1) is used to cool down or warm up by using a fluid (3), for example, air, it can be folded because it is made of ultra-light material, it is stored folded in a flat package. Once the package is open, the blanket is unfold, the sheet that is contact with the patient is identified, the coverings that cover the adhesive material are removed and the blanket is carefully installed on the patient's neck, making sure that it is perfectly adhered.

In a further embodiment, when the heat exchange fluid is a liquid fluid, for example water, the cervical blanket (1) adopts the shape of a hollow, plastic structure, which adapts to the surface under the neck and shoulders of the patient and through circulation of a liquid fluid at different temperatures therein, heat is transferred or heat losses are increased by convection. It can be sterilized to be reused, not disposable. In this case, the cervical blanket (1) is a tubular structure comprising a fluid inlet on the right hand side and an outlet on the left hand side wherein at the portion that is in contact with the patient the single inlet piping is subdivided into a series of ramifications intended to supply the water fluid in a laminar manner like a mesh in order to cover the largest skin surface to converge, at the water outlet zone, into one single duct that returns water, that already transferred heat to a patient, to the heat exchanger. The cervical blanket (1) can be adapted but can not be dilated easily so that even with larger fluid fluxes, it does not become globular in shape. It is completely coated by a plastic coating with the shape of a patient's neck and shoulder surface wherein the external face acts as a thermal insulator (to prevent effectiveness loss and to only allow patient temperature exchange and not room temperature exchange) and the internal face comprises a mechanism for adapting to the patient's neck. To optimize the convection temperature transfer, when a liquid fluid circulates through the ducts (2) of the cervical blanket (1) a perfect contact between surfaces is required and by means of a specific interface, the cervical blanket can be adapted to the patient's neck surface without exerting uneven pressure on different areas and without occluding venous circulation. This interface consists of an air bag located between the surface of the skin and the surface of the blanket lines transporting liquid fluid. This bag comprises a connector that can be adapted to any vacuum systems available in every medical center.

The cervical blanket (1) may have different sizes and shapes so that it can be adapted for use in pediatric or adult patients.

In one embodiment, when the fluid (3) used is air, the heat exchanger (4) is an air conditioner. In this embodiment, the air conditioner is an electric portable unit (equipped with wheels) which comprises a rechargeable battery and plug to be connected to the current electric grid. At the rear of the heat exchanger (4) a power supply connector outlet is located.

In a further embodiment of the invention, the fluid (3) is a liquid fluid. In a more particular embodiment, the fluid (3) is water. In this case, the heat exchanger (4) corresponds to a portable structure operated by batteries or connected to power supply and comprises a water cooler or heater together with a centrifugal pump which propels liquid fluids; it comprises wheels and a plastic tank for storage, with volumes ranging from 1 to 20 liters. At the rear, the heat exchanger comprises two liquid fluid inlets and two outlets which are pressure connectors for the cervical blanket system (1).

In a particular embodiment, when the fluid (3) is air, means of connection (5) from the air conditioner outlet at the rear of the heat exchanger (4) are circular in shape and they are fixedly connected to a coaxial, circular corrugated pipe that transports air to the cervical blanket (1). The means of connection (5) at distal end thereof comprise a conical connector to fit the cervical blanket connector. The means of connection (5) by being coaxial allows the air transported therein to maintain the conditioner outlet temperature unchanged (selected at the console) regardless of the pipe length.

In a further embodiment, when fluid is liquid, the means of connection (5) correspond to 1 to 10 meter lines and comprise an adapter located at the heat exchange (4) liquid fluid outlet and an adapter located at the cervical blanket (1) liquid fluid inlet and outlet. The means of connection (5) corresponding to a pipe wherein liquid fluid is transported are made of plastic, hypoallergenic material which is thermostable at temperatures from 10° C. to 43° C.

The controller (7) comprises a control-console wherein a power button is located; a temperature selector to select the temperature at which the fluid will be heated or cooled; a pump flow selector; the flow being expressed for example as liters per minute; and a display that provides information regarding the temperature selected at the temperature selector, the liquid fluid outlet temperature through the heat exchanger outlet duct, the patient's temperature, the rate at which the pump operates, for example, in revolutions per minute, and the fluid flow per minute.

The patient's temperature sensor (6) corresponds to an esophageal thermometer equipped with a long cable which extends from the patient's esophagus and it is connected to the controller (7).

Method for Rising or Lowering a Patient's Temperature by Using the System of the Present Invention

Once the cervical blanket is installed on the patient (1), a body temperature sensor is inserted (6), for example, an esophageal thermometer connected to the controller (7). The console is switched on and the patient target temperature is selected. The means of connections (5) are connected from the heat exchanger (4) to the cervical blanket (1) making sure that the cervical blanket (1) fluid inlet does not collapse. Particularly, when the fluid (3) is air, upon operating the heat exchanger (4), it can be observed how the blanket is inflated, and then it is covered by a fabric sheet to make better use of the air convection currents.

In a particular embodiment, when the fluid (3) is liquid, the proper size of the blanket is chosen. A temperature sensor (6), for example, an esophageal thermometer which is inserted into the patient is connected to the controller (7). The cervical blanket (1) is placed over the patient's neck allowing the blanket to accommodate without exerting pressure. Then, the blanket must be best adapted to the neck by using both hands and when this adaptation is considered appropriate without having neck compressed areas, vacuum is connected to the bag which is in contact with the patient's skin. After maximum vacuum is applied to the bag which is in contact with the patient, it has to be verified that the structure is adapted to the patient's neck, with “memory”. In order to optimize temperature transfer, a gel is then applied between the skin and the cervical blanket for maximum contact between them.

The heat exchanger motor is switched on, and the following is selected: i) water temperature, ii) patient's target temperature and iii) fluid flow will be modified according to patient cooling or warming rate.

Unlike most of the effective cooling systems, this is a novel noninvasive patient cooling and warming system and it is more efficient for temperature transfer than the existing water mattresses which only cover patient's thoracic and back surfaces; some surfaces may not be in direct contact and the laminar flow over the skin is not achieved.

### APPLICATION EXAMPLES

In the specialized literature, systems for managing temperature through convection comprise cooling average rates from 2 to 1.5° C. an hour (Therapeutic hypothermia and controlled normothermia in the intensive care unit: Practical considerations, side effects, and cooling methods. Crit Care Med 2009 Vol. 37, No. 3) which does not differ from the present invention.

The cooling rate achieved by the system and method of the present invention is rather higher due to the closeness to neck blood.

In one embodiment of the present invention, when the heat exchange fluid is water, cooling rate is around 2° C. or higher per hour.

In the case of the embodiment of the invention using air as the fluid, the transfer rate is higher, with rates around 3 to 5° C. per hour.

However, in addition to achieve similar or higher cooling rates compared to those described in the literature for commercially available products, the present invention has the following comparative advantages with regard to other methods, namely:

1. The existing products that modify body temperature cover large areas of the skin, either thorax or the entire head resulting in a difficult daily management of patients.

Patients with head trauma or brain damage following cardiac arrest require brain signs monitoring which are detected through elements in contact with the scalp, for example, an electroencephalogram, or elements that penetrate the cranial bone to reach brain structures such as Camino intracranial pressure catheters with Lycox. If the technology for modifying the patient's temperature obstructs the access to the scalp or cranium, this must be removed while monitoring brain sings leading to a discontinuation of the hypothermia therapy. The present invention which is limited to patient's neck and shoulders does not obstruct the access to the patient's mouth, airways or cranium.

On the other hand, products for modifying patient's temperature that have to be in contact with patient's torso covering large contact surfaces obstruct the inspection of the thorax and abdomen or the access thereto, as well as important access sites for medical exams such as echocardiograms, electrocardiograms, abdominal echotomography or procedures such as surgeries or punctures.

These transient interruptions are not devoid of complications, specially, with regard to patient overheating since the initial condition may be worsened.

Finally, punctures must be done on the neck when admitted to the Intensive Care Unit in order to introduce catheters that remain in situ even two weeks so that once installed and covered by the present invention, they do not have to be removed until two week later, i.e., when hypothermia treatment is no longer required.

2. The present invention is easy to install.

3. The closeness to structures such blood vessels to achieve blood cooling makes the present invention unique. Unlike the present invention which only covers the neck surface, prior art products for heat transfer are less effective since they must cover large areas of the thorax and abdomen skin surfaces located far apart.

1. Noninvasive system for rising or lowering a patient's body temperature in a controlled manner, wherein the system comprises:
a. a cervical blanket (1) comprising ducts (2) through which a fluid circulates (3);
b. a fluid (3) capable of transferring heat from and to external bodies;
c. a heat exchanger (4) which rises or lowers the fluid temperature that circulates through cervical blanket ducts;
d. means of connection (5) between ducts (2) of the cervical blanket (1) an the heat exchanger (4);
e. a body temperature sensor (6);
f. a controller (7) which rises or lowers fluid temperature according to a predetermined temperature and according to the temperature measured by the body temperature sensor (6).

2. Noninvasive system for rising or lowering a patient's body temperature in a controlled manner according to claim 1, wherein the controller (7) comprises a control-console where the power button is located; a temperature selector to select the temperature at which the fluid will be heated or cooled; a pump flow selector; the flow being expressed for example as liters per minute; and a display that provides information regarding the temperature selected at the temperature selector, the liquid fluid outlet temperature through the heat exchanger outlet duct, the patient's temperature, the rate at which the pump operates, for example, in revolutions per minute, and the fluid flow per minute.

3. Noninvasive system for rising or lowering a patient's body temperature in a controlled manner according to claim 1, wherein the patient's temperature sensor (6) corresponds to an esophageal thermometer equipped with a long cable which extends from the patient's esophagus and it is connected to the controller (7).

4. Noninvasive system for rising or lowering a patient's body temperature in a controlled manner according to claim 1, wherein the fluid (3) is air.

5. Noninvasive system for rising or lowering a patient's body temperature in a controlled manner according to claim 1, wherein the fluid (3) is a liquid.

6. Noninvasive system for rising or lowering a patient's body temperature in a controlled manner according to claim 1, wherein the fluid (3) is water.

7. Noninvasive system for rising or lowering a patient's body temperature in a controlled manner according to claim 1, wherein the cervical blanket (1) is a disposable inflatable device and it is manufactured from two thin synthetic sheets, one external and one internal, made of latex-free hypoallergenic material, fixed together along the free edge thereof so that between them there exists a virtual space, wherein both thin sheets are rectangular in shape; wherein one of the sheets corresponds to the internal sheet and it is made of a mixture of cellulose and a plastic polymer, and wherein the internal sheet, comprises on the external surface two self-adhesive bands arranged lengthwise and further comprises fenestrations or vents that enable air to flow; and wherein the external thin sheet, in the same shape as the internal sheet, is an appendix which emerges from the device lateral surface relative to neck with a conical adaptor where a means of connection (5) coming from the heat exchanger (4) is connected.

8. Noninvasive system for rising or lowering a patient's body temperature in a controlled manner according to claim 7, wherein both sheets, internal and external, are attached not only along the free edge thereof but also in a cross-sectional manner via perpendicular sheets attached to both sheets so that incomplete septa are produced so the air travels freely across the whole length of both sheets serving as an anchor to prevent the device, once inflated, from having a globular shape.

9. Noninvasive system for rising or lowering a patient's body temperature in a controlled manner according to claim 1, wherein the heat exchanger (4) is an air conditioner which is an electric portable unit (equipped with wheels) which comprises a rechargeable battery and plug to be connected to the current electric grid, and wherein at the rear of the heat exchanger (4) a power supply connector outlet is located.

10. Noninvasive system for rising or lowering a patient's body temperature in a controlled manner according to claim 1, wherein the means of connection (5) are circular in shape and they are fixedly connected to a coaxial, circular corrugated pipe that transports air to the cervical blanket (1), and wherein the means of connection (5) at the distal end thereof comprise a conical connector to fit the cervical blanket (1) connector.

11. Noninvasive system for rising or lowering a patient's body temperature in a controlled manner according to claim 1, wherein the cervical blanket (1) is a hollow, plastic, sterilizable structure, wherein an internal face and an external face are defined, and comprises a tubular structure comprising a fluid inlet (14) and an outlet (15), and wherein at the internal face piping is subdivided into a series of ducts (2) so that the water flow is supplied in a laminar manner, and wherein at the water outlet zone such ducts (2) converge into a single duct which returns the fluid (3) to the heat exchanger.

12. Noninvasive system for rising or lowering a patient's body temperature in a controlled manner according to claim 11, wherein the cervical blanket (1) is completely coated by a plastic coating with the shape of a patient's neck and shoulder surface wherein the external face acts as a thermal insulator and the internal face comprises a mechanism (18) for adapting to the patient's neck.

13. Noninvasive system for rising or lowering a patient's body temperature in a controlled manner according to claim 1, wherein the means of connection (5) correspond to 1 to 10 meter lines and comprise an adaptor located at the heat exchange (4) liquid fluid outlet and an adaptor located at the cervical blanket (1) liquid fluid outlet and inlet, and wherein the means of connection (5) comprise a pipe wherein fluid (3) is transported, and wherein they are made of plastic, hypoallergenic, and temperature stable material at temperatures ranging from 10° C. to 43° C.

14. Noninvasive system for rising or lowering a patient's body temperature in a controlled manner according to claim 1, wherein the heat exchanger (4) is a portable structure operated by batteries or connected to power supply and comprises a liquid fluid heater and cooler, together with a centrifugal pump for propelling liquid fluids, and it comprises wheels and a plastic tank for storage, and wherein at the rear face it comprises two liquid fluid inlets and two outlets which are pressure connectors for the cervical blanket system (1).

15. Method for rising or lowering a patient's body temperature in a noninvasive and controlled manner wherein the cervical blanket (1) is placed over the patient's neck allowing the blanket to accommodate without exerting pressure; the blanket is then best adapted to the neck by using both hands and when this adaptation is considered appropriate without having neck compressed areas, a body temperature sensor (6) is inserted into the patient, wherein such sensor is connected to a controller (7), the controller sensor is switched on (7) and the patient's target temperature is selected, the means of connection (5) are connected from the heat exchanger (4) to the cervical blanket (1) making sure the cervical blanket (1) fluid inlet does not collapse.

16. Method for rising or lowering a patient's body temperature in a noninvasive and controlled manner according to claim 15, wherein the cervical blanket is covered by a fabric sheet (12) to make best use of convection currents.

17. Method for rising or lowering a patient's body temperature in a noninvasive and controlled manner according to claim 15, wherein a vacuum pump is connected to the bag (17) which is in contact with the patient's skin, and it is verified that the structure is adapted to the patient's neck, then, in order to optimize heat transfer, a gel is applied between the skin and the cervical blanket for maximum contact between them.

18. Noninvasive system for rising or lowering a patient's body temperature in a controlled manner according to claim 4, wherein the cervical blanket (1) is a disposable inflatable device and it is manufactured from two thin synthetic sheets, one external and one internal, made of latex-free hypoallergenic material, fixed together along the free edge thereof so that between them there exists a virtual space, wherein both thin sheets are rectangular in shape; wherein one of the sheets corresponds to the internal sheet and it is made of a mixture of cellulose and a plastic polymer, and wherein the internal sheet, comprises on the external surface two self-adhesive bands arranged lengthwise and further comprises fenestrations or vents that enable air to flow; and wherein the external thin sheet, in the same shape as the internal sheet, is an appendix which emerges from the device lateral surface relative to neck with a conical adaptor where a means of connection (5) coming from the heat exchanger (4) is connected.

19. Noninvasive system for rising or lowering a patient's body temperature in a controlled manner according to claim 4, wherein the heat exchanger (4) is an air conditioner which is an electric portable unit (equipped with wheels) which comprises a rechargeable battery and plug to be connected to the current electric grid, and wherein at the rear of the heat exchanger (4) a power supply connector outlet is located.

20. Noninvasive system for rising or lowering a patient's body temperature in a controlled manner according to claim 4, wherein the means of connection (5) are circular in shape and they are fixedly connected to a coaxial, circular corrugated pipe that transports air to the cervical blanket (1), and wherein the means of connection (5) at the distal end thereof comprise a conical connector to fit the cervical blanket (1) connector.
